(19) United States
(12) Patent Application Publication
(10) Pub. No.: US 2002/0197226 A1
(43) Pub. Date: Dec. 26, 2002

(54) Composition comprising a polysaccharide grafted with a polysiloxane, and non-cellulosic, non-fixing thickener

(76) Inventors: Franck Giroud, Clichy (FR); Christine Dupuis, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 10/094,811

(22) Filed: Mar. 12, 2002

(30) Foreign Application Priority Data
Mar. 13, 2001 (FR) 01 03403

(51) Int. Cl. A61K 7/06; A61K 7/11

(52) U.S. Cl. 424/70.12

(57) ABSTRACT

A cosmetic hair composition, such as a gel, comprising: (a) at least one polymer comprising a polysaccharide skeleton grafted with at least one group comprising at least one polysiloxane; and (b) at least one non-cellulosic, non-fixing thickener. The invention also relates to a method of making the composition, a cosmetic hair process comprising the application of this composition to the hair, and also to methods for fixing and/or holding the hairstyle.

The invention relates to a cosmetic hair composition, such as a gel, comprising: (a) at least one polymer comprising a polysaccharide skeleton grafted with at least one group comprising at least one polysiloxane, and (b) at least one non-cellulosic non-fixing thickener. The invention also relates to a cosmetic hair process comprising the application of this composition to the hair, and also to its use for fixing and/or holding the hairstyle.

For the purposes of the present invention, the term “gel” means a composition that is initially liquid, thickened by means of an agent with thickening and/or gelling properties, and finally having a minimum viscosity of 160 cps, such as a viscosity of 250 cps at 25° C. (Rhéomat 180 viscometer—spindle 2—reading after 30 seconds).

The expression “non-cellulosic thickener” means any compound, free of cellobiose units, capable of increasing the viscosity of a cosmetic composition.

The expression “non-fixing thickener” means any thickening compound which by itself is insufficient, when it is present in a cosmetic hair composition, to fix and/or hold the hairstyle in a desired shape.

Products for fixing and/or holding the hairstyle that are in various forms, such as in the form of gels, are known. Although these products often allow a long-lasting fixing of the hairstyle, they nevertheless can have various drawbacks.

Many consumers make the criticisms that the gel remains stuck to the hands during application, that its drying time on the hair is long, which may result in a collapse of the hairstyle if the gel is applied as a finish to dry hair, and that it can give hair an artificial appearance, such as an unnatural sheen.

Gels have also been criticized for forming a mousse during their application, which can be detrimental to the aesthetics and to the ease of application of the product.

To overcome the above problems, it has been previously proposed to produce cosmetic hair products comprising a fixing polymer and a thickener with optional additives. However, the compositions known to date comprising these constituents are still not entirely satisfactory, since the hairstyle can still lack a natural look and since the gels can continue to be too sticky or to form a mousse on the hands during application. The compositions may also dry too slowly.

Workers in the art have sought a cosmetic hair composition, such as in gel form, which can overcome at least one of the drawbacks of the prior art compositions. There remains a need for a composition that can spread uniformly, without giving rise to the formation of a mousse in its application, and which can give satisfactory cosmetic results.

The inventors have discovered, surprisingly and unexpectedly, that by combining suitable polymers with non-cellulosic, non-fixing thickeners, it is possible to solve at least one of the problems posed above.

One subject of the invention is a cosmetic composition comprising, in a cosmetically acceptable medium, (a) at least one polymer comprising a polysaccharide skeleton grafted with at least one group comprising at least one polysiloxane, and (b) at least one non-cellulosic, non-fixing thickener.

Another subject of the invention comprises a method of preparing a composition, comprising dissolving, in an organic solvent, (a) a polysaccharide containing carboxyl groups, and (b) a polysiloxane containing an epoxy end group and corresponding to formula (I), defined below, for a time and under conditions sufficient to obtain a polysaccharide and polysiloxane mixture. The method further comprises heating the mixture to a temperature of between 60 and 200° C. inclusive; reacting the carboxyl groups of the polysaccharide with the epoxy groups of the polysiloxanes for a time and under conditions sufficient to form a polymer comprising a polysaccharide skeleton grafted with at least one group comprising at least one polysiloxane; and adding at least one non-cellulosic, non-fixing thickener to the grafted polymer to prepare said composition.

Another subject of the present invention relates to a cosmetic process, such as a hair process, for fixing and/or holding a hairstyle, comprising applying to hair, an effective amount of a composition comprising, in a cosmetically acceptable medium (a) at least one polymer comprising a polysaccharide skeleton grafted with at least one group comprising at least one polysiloxane, and (b) at least one non-cellulosic, non-fixing thickener.

Yet another subject of the invention relates to a method for holding and/or shaping a hairstyle, comprising applying to hair, an effective amount of a composition comprising, in a cosmetically acceptable medium (a) at least one polymer comprising a polysaccharide skeleton grafted with at least one group comprising at least one polysiloxane, and (b) at least one non-cellulosic, non-fixing thickener.

The polymers comprising at least one polymer comprising a polysaccharide skeleton grafted with at least one group comprising at least one polysiloxane, in accordance with the present invention, comprise a principal chain formed from polysaccharide(s), onto which is grafted, and also optionally attached to at least one of its ends, at least one group comprising a polysiloxane.

These polymers comprising a polysaccharide skeleton grafted with at least one group comprising at least one polysiloxane, may be obtained according to any means known to those skilled in the art, such as by a reaction between: (i) a starting polysiloxane macromer functionalized on the polysiloxane chain; and (ii) at least one polysaccharide, which can be functionalized with a group that is capable of reacting with the functional group(s) borne by the said silicone to form a covalent bond.

Exemplary polysaccharide grafted polymers are described in U.S. Pat. No. 6,066,727, by the company Shin-Etsu, the disclosure of which is specifically incorporated by reference herein. These are copolymers obtained by reaction between a polysaccharide containing carboxyl groups and a polysiloxane containing an epoxy end group, in an organic solvent, optionally in the presence of a catalyst.

In one embodiment, the process for preparing these polymers comprises:

(a) dissolving, in an organic solvent, (a) a polysaccharide containing carboxyl groups, and (b) a polysiloxane containing an epoxy end group, and corresponding to formula (I) of a polysiloxane below:

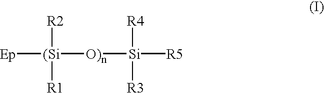

in which

n is an integer between 3 and 500 inclusive,

R₁, R₂, R₃, R₄ and R₅ are chosen, independently of each other, from monovalent C₁ to C₁₀ hydrocarbons and monovalent C₁ to C₁₀ halohydrocarbons, and

Ep is 2-(3,4-epoxycyclohexyl)ethyl; and

(b) heating this mixture of polysaccharides and polysiloxanes to a temperature between 60 and 200° C. inclusive, so as to make the carboxyl groups of the polysaccharides react with the epoxy groups of the polysiloxanes.

Exemplary polysaccharides suitable for carrying out step (a) of this process, include polysaccharides containing at least one group chosen from carboxylic, benzoyl and succinoyl groups, such as hydroxypropylmethylcellulose phthalate and hydroxypropylmethylcellulose acetate succinate, and also include carboxymethylethylcellulose, and the polysaccharide pullulan acetate phthalate.

R₁, R₂, R₃, R₄ and R₅ can be chosen, independently of each other, from methyl, ethyl, propyl and butyl groups, C₃ to C₈ cycloalkyl groups such as cyclopentyl and cyclohexyl radicals, aryl groups such as phenyl and tolyl radicals, C₃ to C₈ aralkyl groups, such as benzyl and phenethyl radicals, and alkenyl groups, such as vinyl and allyl groups. These monovalent hydrocarbon radicals may optionally be totally and partially substituted, such as with a halogen atom, for example, with chloromethyl and 3,3,3-trifluoropropyl radicals.

Exemplary organic solvents suitable for performing step (a) of the process, include solvents chosen from ketones, for instance acetone, and cyclohexanone.

Step (b) can be carried out with stirring, optionally under an inert atmosphere.

The composition can comprise, as a relative percentage by weight of the composition, from 0.1% to 20% of grafted polysaccharide, such as from 0.5% to 10% of the grafted polysaccharide.

The composition can comprise, as a relative percentage by weight of the composition, from 0.01% to 20% of non-cellulosic thickener, such as from 0.05% to 10% of non-cellulosic thickener.

The non-cellulosic thickener can be chosen from: (i) natural polymers and modified natural polymers; (ii) crosslinked copolymers derived from monomers chosen from acrylic and methacrylic acid; (iii) crosslinked thickening polyacrylamides; and (iv) associative polymers comprising at least one hydrophilic unit and at least one fatty chain.

According to the invention, the expression “modified natural thickener” (i) means any thickening polymer obtained by chemical modification starting with the natural polymer itself.

Natural thickeners and modified natural thickeners (i) that are suitable for the invention include those chosen from scleroglucan gum, gellan gum, rhamsan gum, alginates, karaya gum, carob flour, modified and unmodified guar gums and derivatives thereof, for instance hydroxypropylguar. Guar gum and its derivatives (hydroxypropylguar) and gums of microbial natural origin, such as xanthan gum and scleroglucan gum can be used as well.

Exemplary crosslinked copolymers (ii) include acrylic acid/ethyl acrylate copolymers and carboxyvinyl polymers, such as “carbomers” (CTFA) sold by the company Goodrich under the name Carbopol.

Exemplary crosslinked thickening polyacrylamides (iii) may be chosen from:

crosslinked 2-acrylamido-2-methylpropane sulphonic homopolymers,

optionally crosslinked copolymers derived from acrylamide and ammonium acrylate,

crosslinked copolymers derived from (a) monomers chosen from acrylamides, methacrylamides, and (b) methacryloyloxyethyltrimethylammonium chloride,

partially and totally neutralized crosslinked copolymers derived from acrylamide and 2-acrylamido-2-methylpropanesulphonic acid.

As crosslinked acrylamide/ammonium acrylate copolymers used in accordance with the present invention, mention may be made of acrylamide/ammonium acrylate (5/95 by weight) copolymers crosslinked with a crosslinking agent containing olefinic polyunsaturation, such as divinylbenzene, tetraallyloxyethane, methylenebisacrylamide, diallyl ether, polyallylpolyglyceryl ethers and allylic ethers of alcohols of the sugar series, such as erythritol, pentaerythritol, arabitol, mannitol, sorbitol and glucose.

Similar copolymers are described and prepared in French patent FR-2 416 723 and U.S. Pat. Nos. 2,798,053 and 2,923,692, the disclosures of which are specifically incorporated by reference herein.

This type of crosslinked copolymer can be in the form of a water-in-oil emulsion comprising 30% by weight of the said copolymer, 25% by weight of liquid paraffin, 4% by weight of a mixture of sorbitan stearate and of a hydrophilic ethoxylated derivative, and 41% by weight of water. Such an emulsion is sold under the name “Bozepol C” by the company Hoechst.

The copolymers derived from acrylamide and 2-acrylamido-2-methylpropanesulphonic acid used in accordance with the present invention include those copolymers crosslinked with a compound containing olefinic polyunsaturation, such as those mentioned above, and partially and totally neutralized with a neutralizer, such as those chosen from sodium hydroxide, potassium hydroxide, aqueous ammonia and an amine such as triethanolamine and monoethanolamine.

They may be prepared by copolymerizing acrylamide and sodium 2-acrylamido-2-methylpropane sulphonate via a free radical route by using initiators of the type such as azobisisobutyronitrile and by precipitation from an alcohol such as tert-butanol.

Copolymers obtained by copolymerization of 70 to 55 mol % of acrylamide and 30 to 45 mol % of sodium 2-acrylamido-2-methylpropane sulphonate can also be used. The crosslinking agent can be used at concentrations of from 10⁻⁴ to 4×10⁻⁴ mol per mole of the monomer mixture.

These copolymers can be incorporated into the compositions of the invention in the form of water-in-oil emulsions containing from 35% to 40% by weight of this copolymer, from 15% to 25% by weight of a mixture of C₁₂-C₁₃ isoparaffinic hydrocarbons, from 3% to 8% by weight of polyethylene glycol lauryl ether containing 7 mol of ethylene oxide, and water. Such an emulsion is sold under the name “Sepigel 305” by the company SEPPIC.

The crosslinked copolymer derived from acrylamide and methacryloyloxyethyltrimethylammonium chloride used according to the invention can be obtained by copolymerization of acrylamide and of dimethylaminoethyl methacrylate quaternized with methyl chloride, followed by crosslinking with an olefinically unsaturated compound, such as methylenebisacrylamide.

The non-crosslinked copolymers derived from methacrylamide and methacryloyloxyethyltrimethylammonium chloride are, for example, the products sold under the trade names Rohagit KF 400 and KF 720 by the company Rohm & Haas.

The associative polymers (iv) of the invention may be chosen from nonionic, cationic, anionic and amphoteric polymers. They may, for example, belong to the polyurethane class.

The associative polyurethanes are block copolymers comprising in the chain both hydrophilic sequences usually of polyoxyethylenated nature and hydrophobic sequences which may be chosen from aliphatic chain units, cycloaliphatic chain units, and aromatic chain units.

These polymers can, for example, comprise at least two hydrocarbon-based lipophilic chains containing from 6 to 30 carbon atoms, separated by a hydrophilic sequence. The hydrocarbon-based chains can be chosen from pendent chains and chains at the end of the hydrophilic sequence. It is possible for at least one pendent chain to be present. In addition, the polymer can comprise a hydrocarbon-based chain at one or both ends of a hydrophilic sequence.

The polymers can be sequenced in a form chosen from triblock and multiblock forms. The hydrophobic sequences can thus be at each end of the chain (for example: triblock copolymer with a hydrophilic central sequence) or distributed both at the ends and in the chain (for example multiblock copolymer). The polymers can also be chosen from graft polymers and starburst polymers.

The polymers can, for example, be triblock copolymers whose hydrophilic sequence is a polyoxyethylenated chain comprising from 50 to 1 000 oxyethylene groups. The associative polyurethanes can comprise a urethane linkage between the hydrophilic sequences.

By extension, polymers whose hydrophilic sequences are linked by other chemical linkages to the lipophilic sequences also are examples of useful associative polyurethanes.

As examples of associative polymers which can be used in the invention, mention may be made of the polymer C₁₆-OE₁₂₀-C₁₆ sold by the company Hüls (under the name Serad FX1100, this molecule containing a urethane function and having a weight-average molecular weight of 1 300), OE being an oxyethylene unit. Associative polymers which can also be used are Rheolate 205 containing a urea function, sold by the company Rheox, and Rheolate 208 and 204. These associative polyurethanes are sold in pure form.

The product DW 1206B from Rhom & Haas, containing a C₂₀ alkyl chain and a urethane linkage, sold at a concentration of 20% solids in water, can also be used.

Solutions and dispersions of these polymers in water and in aqueous-alcoholic medium can also be used. Examples of such polymers which may be mentioned are Serad FX1010 and Serad FX1035 sold by the company Hüls, and Rheolate 255, Rheolate 278 and Rheolate 244 sold by the company Rheox. The products DW 1206F and DW 1206J can also be used, as can Acrysol RM 184 and Acrysol 44 from the company Rhom & Haas.

The polymers which can be used in the invention include those described in the article by G. Fonnum, J. Bakke and Fk. Hansen—Colloid Polym. Sci 271, 380.389 (1993), the disclosure of which is specifically incorporated by reference herein.

The associative polyurethanes (iv) may be chosen from:

polyurethane polyethers (a) which can be obtained by polycondensation of at least three compounds comprising (i) at least one polyethylene glycol comprising from 50 to 500 mol of ethylene oxide, (ii) at least one C₈-C₃₀ fatty alcohol and (iii) at least one diisocyanate and,

polyurethane polyethers (b) which can be obtained by polycondensation of at least three compounds comprising (i) at least one polyethylene glycol comprising from 50 to 500 mol of ethylene oxide, (ii) at least one C₈-C₃₀ fatty alcohol other than that of the polyurethane polyether (a) and (iii) at least one diisocyanate.

Polyurethane polyethers (a) and (b) can be used, for example, those polyethers for which the polyethylene glycol is present in amounts such as 150 and 180 mol of ethylene oxide.

Polyurethane polyethers (a) and (b) for which the diisocyanate is methylenebis(4-cyclohexyl isocyanate) can be used.

Another exemplary composition includes polyurethane polyethers (a) obtained by polycondensation of at least three compounds comprising a polyethylene glycol containing ethylene oxide present in an amount chosen from 150 and 180 mol, stearyl (C18) alcohol and methylenebis(4-cyclohexyl isocyanate), and polyurethane polyethers (b) obtained by polycondensation of at least three compounds comprising a polyethylene glycol containing 150 or 180 mol of ethylene oxide, decyl (C10) alcohol and methylenebis(4-cyclohexyl isocyanate).

Among the polyurethane polyethers (a), mention may be made of the product sold by the company Rohm & Haas under the trade name Aculyn 46, which is a polycondensate comprising at least, as elements, a polyethylene glycol containing 150 or 180 mol of ethylene oxide, stearyl alcohol and methylenebis(4-cyclohexyl isocyanate) (SMDI), at 15% by weight in a matrix of maltodextrin (4%) and water (81%).

Among the polyurethane polyethers (b), mention may be made of the product sold by the company Rohm & Haas under the trade name Aculyn 44, which is a polycondensate comprising at least, as elements, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%).

Viscophobe DB 1000 (Union Carbide) may also be used as an associative polyurethane.

As other associative polymers which may be used, mention may be made of those copolymers chosen from acrylic acid/C10-C30 alkyl acrylate and methacrylic acid/C10-C30 alkyl acrylate copolymers, such as Pemulen TR1 from Goodrich.

The cosmetically acceptable medium can comprise at least one solvent chosen from water and cosmetically acceptable solvents, such as alcohols and water-solvent mixtures. Exemplary solvents include C₁-C₄ alcohols, aliphatic and aromatic ketones, esters of C₁-C₈ and C₉-C₂₀ acids and of C₁-C₈ and C₉-C₂₀ alcohols, pentane, heptane, polyols, polyol ethers and isodecane.

Other exemplary solvents include at least one alcohol chosen from ethanol and isopropanol.

The ratio between the relative concentration by weight of the polymers containing polysaccharide skeletons, and the relative concentration by weight of the non-cellulosic non-fixing thickeners, can range from 0.01:1 to 100:1, such as ratios ranging from 0.05:1 to 20:1 and also from 0.1:1 to 10:1.

The composition of the invention may also contain at least one additive chosen from: anionic, cationic, nonionic and amphoteric surfactants; fragrances; screening agents; preserving agents; proteins; provitamins; ceramides; polymers other than those of said at least one polymer and said at least one thickener; plant, mineral and synthetic oils; polyols chosen from glycols and glycerol; silicones; fatty alcohols; and any other additive conventionally used in cosmetic compositions.

A person skilled in the art can take care to select the optional compound(s) to be added to the composition according to the invention such that the advantageous properties intrinsically associated with the composition in accordance with the invention are not adversely affected by the envisaged addition.

The compositions in accordance with the invention may be applied to the skin, the nails, the lips, the hair, the eyebrows, and the eyelashes.

The compositions in accordance with the invention may be suitable for wet or dry hair, as styling products.

The invention will be illustrated more fully with the aid of the non-limiting example which follows.

All the percentages are relative percentage by weight relative to the total weight of the composition, and a.m. means active material.

### EXAMPLE

A gel in accordance with the present invention is prepared from the ingredients listed below.

| Cellulose containing silicone grafts [1] | 4% a.m. |
|---|---|
| Synthalen K [2] | 1.5% a.m. |
| 2-Amino-2-methyl-1-propanol qs pH | 8 |
| Ethanol | 35% |
| Water qs | 100% |

[1] Hydroxypropylmethylcellulose acetate succinate containing polydimethylsiloxane grafts, as synthesized in Example No. 2 of U.S. Pat. No. 6,066,727 by the company Shin Etsu, the disclosure of which is specifically incorporated by reference herein.
[2] Crosslinked polyacrylic acid sold by the company 3V SA.

[1] Hydroxypropylmethylcellulose acetate succinate containing polydimethylsiloxane grafts, as synthesized in Example No. 2 of U.S. Pat. No. 6,066,727 by the company Shin Etsu, the disclosure of which is specifically incorporated by reference herein.

[2] Crosslinked polyacrylic acid sold by the company 3V SA.

No formation of mousse is observed during the application of this gel to the hair.

What is claimed is:

1. A cosmetic composition comprising, in a cosmetically acceptable medium:
(a) at least one polymer comprising a polysaccharide skeleton grafted with at least one group comprising at least one polysiloxane; and
(b) at least one non-cellulosic, non-fixing thickener.

2. The composition according to claim 1, wherein at least one group comprising at least one polysiloxane is attached to at least one end of the polysaccharide skeleton.

3. The composition according to claim 1, wherein the at least one thickener is chosen from: (i) natural polymers and modified natural polymers; (ii) crosslinked copolymers derived from monomers chosen from acrylic and methacrylic acid; (iii) crosslinked thickening polyacrylamides; and (iv) associative polymers comprising at least one hydrophilic unit and at least one fatty chain.

4. The composition according to claim 3, wherein the natural and modified natural polymers (i) are chosen from xanthan gum, scleroglucan gum, gellan gum, rhamsan gum, alginates, karaya gum, carob flour, and guar gums.

5. The composition according to claim 3, wherein the crosslinked copolymers (ii) are chosen from acrylic acid/ethyl acrylate copolymers and carboxyvinyl polymers.

6. The composition according to claim 3, wherein the crosslinked thickening polyacrylamides (iii) are chosen from:
crosslinked 2-acrylamido-2-methylpropane sulphonic homopolymers,
optionally crosslinked copolymers derived from acrylamide and ammonium acrylate,
crosslinked copolymers derived from (a) monomers chosen from acrylamide and methacrylamide, and (b) methacryloyloxyethyltrimethylammonium chloride,
partially and totally neutralized crosslinked copolymers derived from acrylamide and 2-acrylamido-2-methylpropanesulphonic acid.

7. The composition according to claim 3, wherein the associative polymers (iv) are associative polyurethanes.

8. The composition according to claim 1, wherein a ratio of the relative concentration by weight of the at least one polymer to the relative concentration by weight of the at least one thickener ranges from 0.01:1 to 100:1.

9. The composition according to claim 8, wherein the ratio of the relative concentration by weight of the at least one polymer to the relative concentration by weight of the at least one thickener ranges from 0.05:1 to 20:1.

10. The composition according to claim 9, wherein the ratio of the relative concentration by weight of the at least one polymer to the relative concentration by weight of the at least one thickener ranges from 0.1:1 to 10:1.

11. The composition according to claim 1, wherein the composition comprises, as a relative percentage by weight of the composition, from 0.1% to 20% of the at least one polymer.

12. The composition according to claim 11, wherein the composition comprises, as a relative percentage by weight of the composition, from 0.5% to 10% of the at least one polymer.

13. The composition according to claim 1, wherein the composition comprises, as a relative percentage by weight of the composition, from 0.01% to 20% of the at least one thickener.

14. The composition according to claim 13, wherein the composition comprises, as a relative percentage by weight of the composition, from 0.05% to 10% of the at least one thickener.

15. The composition according to claim 1, wherein the composition further comprises at least one additive chosen from: anionic, cationic, nonionic and amphoteric surfactants; fragrances; screening agents; preserving agents; proteins; provitamins; ceramides; polymers other said at least one polymer and said at least one thickener; plant, mineral and synthetic oils; polyols; silicones; fatty alcohols; and any other additive conventionally used in cosmetic compositions.

16. The composition according to claim 15, wherein the polyols comprise at least one polyol chosen from glycols and glycerol.

17. A method of preparing a composition, comprising:
dissolving, in an organic solvent, (a) a polysaccharide containing carboxyl groups, and (b) a polysiloxane containing an epoxy end group and corresponding to formula (I), for a time and under conditions sufficient to obtain a polysaccharide and polysiloxane mixture: 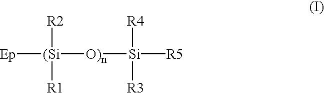
in which:
n is an integer between 3 and 500 inclusive,
R₁, R₂, R₃, R₄ and R₅ are chosen, independently of each other, from monovalent C₁ to C₁₀ hydrocarbons and monovalent C₁ to C₁₀ halohydrocarbons, and
Ep is 2-(3,4-epoxycyclohexyl)ethyl;
heating the mixture to a temperature of between 60 and 200° C. inclusive;
reacting the carboxyl groups of the polysaccharide with the epoxy groups of the polysiloxanes for a time and under conditions sufficient to form a polymer comprising a polysaccharide skeleton grafted with groups comprising at least one polysiloxane; and
adding at least one non-cellulosic, non-fixing thickener to the grafted polymer to prepare said composition.

18. The method according to claim 17, wherein the polysaccharide (a) is chosen from carboxylic, benzoyl and succinoyl groups.

19. The method according to claim 18, wherein the polysaccharide (a) is chosen from hydroxypropylmethylcellulose phthalate, hydroxypropylmethylcellulose acetate succinate, carboxymethylethylcellulose, and pullulan acetate phthalate.

20. The method according to claim 17, wherein R₁, R₂, R₃, R₄ and R₅ are chosen, independently of each other, from methyl, ethyl, propyl, butyl, C₃-C₈ cycloalkyls, aryl, C₃-C₈ aralkyls, and alkenyl groups.

21. A cosmetic hair process for fixing and/or holding a hairstyle, comprising:
applying to hair, an effective amount of a composition comprising, in a cosmetically acceptable medium:
(a) at least one polymer comprising a polysaccharide skeleton grafted with at least one group comprising at least one polysiloxane; and
(b) at least one non-cellulosic, non-fixing thickener.

22. A method for holding and/or shaping a hairstyle, comprising:
applying to the hair, an effective amount of a composition comprising, in a cosmetically acceptable medium:
(a) at least one polymer comprising a polysaccharide skeleton grafted with at least one group comprising at least one polysiloxane; and
(b) at least one non-cellulosic, non-fixing thickener.
